(19) United States
(12) Patent Application Publication
(10) Pub. No.: US 2004/0267360 A1
(43) Pub. Date: Dec. 30, 2004

(54) Alloplastic ligament

(76) Inventor: Jakob Huber, Tutzing (DE)

Correspondence Address: NOTARO AND MICHALOS, 100 DUTCH HILL ROAD, SUITE 110, ORANGEBURG, NY, 10962-2100, US

(21) Appl. No.: 10/850,740

(22) Filed: May 21, 2004

(30) Foreign Application Priority Data
May 26, 2003 (DE) 10323890.5

(51) Int. Cl. A61F 2/08

(52) U.S. Cl. 623/13.12; 623/13.14; 623/13.17

(57) ABSTRACT

An alloplastic ligament has a surface covered by titanium or aluminum oxide ceramic, or by zirconium oxide ceramic or by tantalum, so that the synthetic material of the ligament is not in direct contact with the surrounding body environment. A ligament prosthesis, in particular an artificial cruciate ligament, has ends for securing the alloplastic ligament to the bone, in particular to the femur and the tibia, by a hollow screw of self-cutting outer screw threads for screwing into the bone and with inner screw threads. A sleeve having outer screw threads for screwing into the hollow screw and with a securement of the ligament end on the sleeve is also provided.

The invention relates to an alloplastic ligament of a synthetic material, in particular an artificial cruciate ligament as well as a ligament prosthesis.

Such ligaments and prostheses are disclosed in DE 199 21 781 A1 and DE 101 10 827 A1, U.S. Pat. No. 5,392,302; U.S. Pat. No. 5,152,790, WO 97/36557, U.S. Pat. No. 6,190,411.

The cruciate ligament or ligaments are connective tissues of low elasticity. Braided ligaments are conventionally employed as artificial cruciate ligaments.

The goal and task of the invention is proposing further improvements of the known prostheses and ligaments.

This task is solved according to the invention thereby that in the case of an alloplastic ligament of synthetic material the surface of the ligament is completely, or at least locally, furnished with and encompassed by titanium or aluminum oxide ceramic or zirconium oxide ceramic or tantalum, such that the synthetic material ligament does not come into direct contact with the surrounding body environment at least locally, but rather that the contact with the encompassing body environment occurs through the materials titanium, aluminum oxide ceramic, zirconium oxide ceramic or tantalum, which are more compatible with the body.

Consequently, the essential improvement of the known devices takes place thereby that the synthetic ligament is at least locally encompassed, for example coated, in particular nanocoated, with said materials. Therewith these special advantages are attained:

Sheathing the ligament with said material is highly compatible with the body. The contact of endogenous tissue with less body-compatible synthetic material of the ligament can be minimized or prevented entirely.

Sheathing with said materials prevents wear of the synthetic material of the ligament. Such wear of the synthetic material could potentially lead to harmful tissue reactions. This is avoided by sheathing the ligament with said materials.

Due to the extensive wear prevention of the ligament through the sheathing with said material, the device according to the invention is therefore also better suited than the known devices to be applied in younger or highly active persons, for example, in competitive athletes.

The task is also solved thereby that both ends of the ligament comprise a device for securing the alloplastic ligament of synthetic material on bone, in particular on the femur, with a hollow screw comprising self-cutting outer screw threads to be screwed into the bone, with inner screw threads on this hollow screw, with a sleeve comprising outer threads for screwing into the hollow screw and with a securement of the ligament end on the sleeve. Thereby that not only one but both ligament ends are secured with defined clamping, irritations of the periosteum are advantageously avoided. The staples used conventionally can be omitted.

Dependent claims 3 to 11 relate to advantageously structured implementations whose function will be explained with reference to an embodiment example according to the drawing. The figures of the drawing show in detail:

The alloplastic ligament can usefully be secured on one end by defined clamping in the sleeve, and at the end of the sleeve a hexagon is tightly seated serving for screwing into the hollow screw.

The clamping can preferably also be implemented differently. Herein a small sleeve is clamped onto the ligament, optionally utilizing defined indentations of the sleeve against the ligament. When the sleeve is pressed onto the ligament, the inner sleeve is slid above it. Under tensile loading clamping between the small (clamping) sleeve and the inner sleeve takes place. The inner sleeve is subsequently again screwed into the outer sleeve. The femoral and tibial securement takes place analogously to the natural course of the ligament. The sleeves, at whose ends the alloplastic ligament is fastened by definable clamping tension, are comprised of metal, for example titanium, and have outer threads for anchorage in the hollow titanium screw.

The connection of the hexagon with the sleeve is important. The hexagon serves for the simple screwing of the sleeve and of the alloplastic ligament into the hollow titanium screw by means of a hollow hexagonal wrench, which is placed onto the sleeve hexagon. The ligament is also guided in the hollow hexagonal wrench.

The outer screw threads of this sleeve consequently represent the matching counterpiece to the inner screw threads of the hollow titanium screw and thus complement the entire system of securement forming a functional unit.

The round alloplastic ligament of polyethylene terephthalate has a tear strength of for example 4000 N and an extension comparable to that of the natural cruciate ligament. The tear strength of the alloplastic ligament is markedly above that of the natural cruciate ligament (appr. 1300 N), which has the advantage that the alloplastic ligament permits high functional loading.

It is of advantage if in the bore channel of the bone no surface finishing of the synthetic ligament takes place. The surfacing is preferably carried out in the proximity of the free joint. Thereby, if necessary, the ligament can later be replaced without encountering any problem.

Into the femoral bone is screwed the hollow titanium screw, into which, in turn, the sleeve with the alloplastic ligament guided therein is rotated. For the securement in the tibial area, a hollow screw is utilized with a correspondingly larger diameter.

Feasible tibial securements are conical slide-on faces of titanium. These cooperate such that under tensile loading the clamping sleeve clamps the ligament automatically. Through the hollow screw driver in the femoral region, through whose hollow volume the ligament is pulled and which comprises an outer-Allen or hexagonal wrench matching the inner screw, the entire ligament together with the inner screw can be fixated femorally in the outer screw without the synthetic ligament becoming twisted due to the screwing in. The hollow screw driver extends into the sleeve, which is screwed with the outer threads into the hollow titanium screw, which means the self-cutting screw, in which a hexagon is fixedly connected with the sleeve, onto which is placed the inner hexagonal wrench (hollow screw driver).

Exemplary embodiments of the invention will be explained in further detail with reference to the enclosed figures, in which depict:

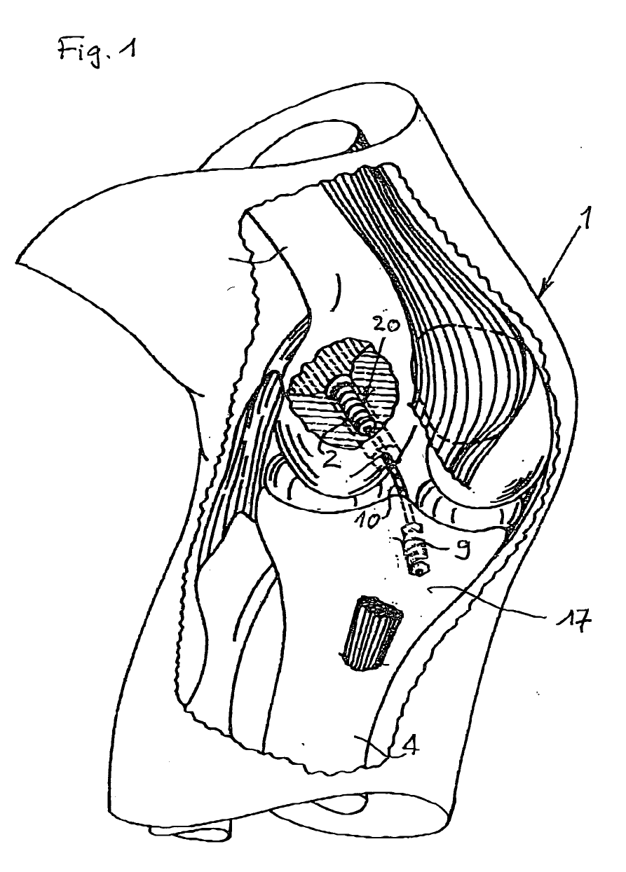

FIG. 1 a knee joint with ligament prosthesis,

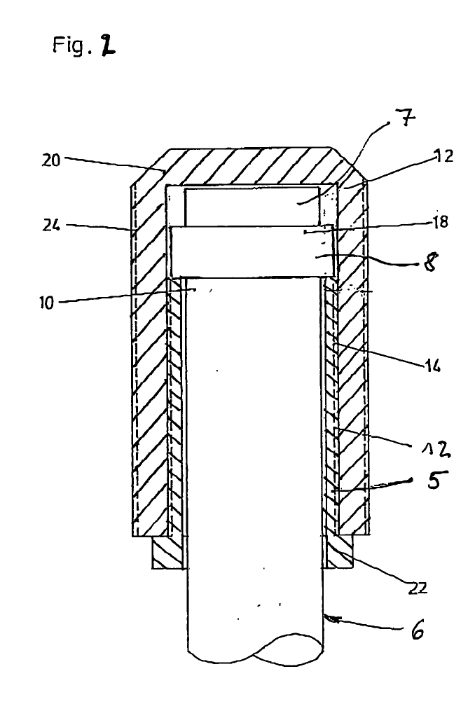

FIG. 2 a securement device for a cruciate ligament on the femur side, and

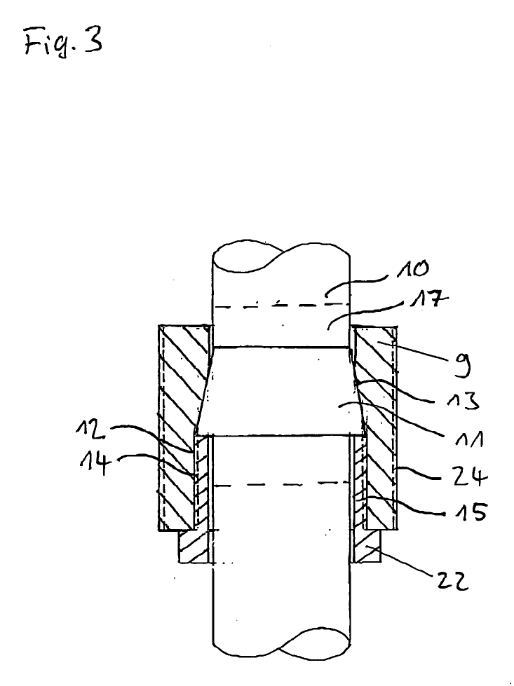

FIG. 3 examples of securements on the tibia side.

FIG. 1 shows a partially cut-away knee joint in perspective view. The ligament prosthesis connects the femur 16 with the tibia 17. The femoral end of a ligament 10 is anchored by means of a hollow screw 20 in the femur 16 and the other end by means of a further hollow screw 9 in the tibia. The femur-side securement is shown in FIG. 2.

The ligament 10, known per se, of polyethylene terephthalate, woven and drawn, is held in an inner sleeve 12 with outer screw threads 14. The inner sleeve 12 is set or screwed into a hollow screw 20, preferably of titanium.

The hollow titanium screw 20 has outer screw threads 24. These outer screw threads are self-cutting and the screw is femorally secured. The hollow titanium screw is first screwed into the femoral bone, and into the hollow titanium screw is screwed the sleeve with the alloplastic ligament. On the inner sleeve 12 is rigidly fastened a hexagon 22. Femoral and tibial securements take place analogously to the natural course of the cruciate ligament. Said hexagon 22 serves for the simple screwing-in of sleeve 12 and of the alloplastic ligament 10 into the hollow titanium screw 20 by means of a hollow hexagonal wrench, which is simply placed onto the sleeve hexagon. In the hollow hexagonal wrench is also guided the ligament.

Outer-thread sleeve 14 and inner-thread hollow screw 20 of titanium are matched to one another (fit one another). The entire system of the femoral securement in this way becomes unified into a functional unit. Sleeve 12 and hexagon 22 form one piece. Novel is inter alia also that, in spite of screwing in the sleeve, the ligament 10 is not being twisted.

A feasible tibial securement is depicted in FIG. 3. The ligament 10 is held between hollow screw 9 and sleeve 22. The self-cutting hollow screw 9 anchors the end of the ligament.

For the femoral securement thus a hollow screw driver with an outer-Allen or hexagonal wrench is used, which fits the inner screw. Of interest is that the entire ligament with inner screw can be femorally fixated in the outer screw without the synthetic ligament becoming twisted through the rotation action and without tensions being exerted onto the bone. If, which per se is not intended, the ligament has to be replaced, a simple option is also available.

With the tibial anchoring, as shown in FIG. 3, the clamping sleeve 11 can initially be freely displaceable on ligament 10. After the hollow screw 9 is screwed in, the cone-shaped clamping sleeve 11 is subsequently slid over the tensioned ligament 10 up into its conical seat in the hollow screw 9. By tightening the sleeve 22 the clamping sleeve is subsequently pressed into the seat, such that the radial pressure generated thereby tightly clamps the ligament 10 permanently due to the conical faces 13 being slid on. The clamping is improved if the section in which the ligament is clamped is reinforced by a pressure sleeve or by other means. The tibial securement for the remainder takes place analogously to the femoral one.

### List of Reference Numbers

1 Knee

2 Ligament prosthesis

3 Femur

4 Tibia

5 Sleeve (femoral)

6 Surface

7 Ligament end

8 Clamping sleeve (femoral)

9 Tibial hollow screw

10 Ligament

11 Clamping sleeve (tibial)

12 Inner sleeve

13 Slide-on face

14 Outer screw threads

15 Sleeve (tibial)

16 Femur

17 Segment

18 Securement

20 Hollow screw

22 Hexagon

24 Outer screw threads (self-cutting)

1. Alloplastic ligament (10) of synthetic material, in particular artificial cruciate ligament, characterized in that the surface (6) of the ligament (10) is completely or at least locally finished with and encompassed by titanium or aluminum oxide ceramic or zirconium oxide ceramic or tantalum, such that the synthetic material ligament is at least locally not in direct contact with the surrounding body environment, but rather the contact with the encompassing body environment occurs with the materials titanium, aluminum oxide ceramic, zirconium oxide ceramic or tantalum, which are more compatible with the body.

2. Ligament prosthesis (2), in particular artificial cruciate ligament, characterized in that both ends comprise a device for securing the alloplastic ligament (10) of synthetic material on the bone, in particular on the femur (3) and the tibia (4), with a hollow screw (20) comprising self-cutting outer screw threads (24) for screwing into the bone (3, 4), with inner screw threads (12) on this hollow screw (20), with a sleeve (5) comprising outer screw threads for screwing into the hollow screw and with a securement (18) of the ligament end on the sleeve.

3. Prosthesis as claimed in claim 2, characterized in that the sleeve (5) comprises outer screw threads (14) opposite the inner screw threads (12) of the hollow screw (20).

4. Prosthesis as claimed in claim 2, characterized in that the ligament (10) is locked at the ligament end (7) in the sleeve (18).

5. Prosthesis as claimed in claim 2, characterized in that the securement (18) of the ligament end (7) on the sleeve (5) comprises a clamping sleeve (8) clamped onto the ligament end and that, on the one hand, the ligament end (7) received with the clamping sleeve (8) in the sleeve (5) and, on the other hand, the sleeve (5) cooperate, such that under tensile loading (10) of the ligament the clamping sleeve (8) is locked in the sleeve (5).

6. Prosthesis as claimed in claim 2, characterized in that the alloplastic ligament (10) is secured on one end (7) through defined locking in the sleeve (5).

7. Prosthesis as claimed in claim 2, characterized in that at the end of the sleeve (5) is fixedly seated a hexagon (22) serving for the screwing into the hollow screw (20), which hexagon avoids a critical deflection through rounding particularly on the inside of the ligament.

8. Prosthesis as claimed in claim 2, characterized in that for the securing against a possible spontaneous loosening between inner sleeve and hollow titanium screw a transverse bolt is introduced into the inner sleeve, in particular toward its end remote from the ligament.

9. Prosthesis as claimed in claim 2, characterized in that the surface (6) of the ligament (10) is completely or at least locally finished with or encompassed by titanium or aluminum oxide ceramic or zirconium oxide ceramic or tantalum, such that the synthetic ligament is at least locally not directly in contact with the surrounding body environment, but rather that the contact with the encompassing body environment occurs with the materials titanium, aluminum oxide ceramic, zirconium oxide ceramic or tantalum which are more compatible with the body.

10. Prosthesis as claimed in claim 2, characterized in that the femoral hollow screw (20) has a smaller diameter than the tibial hollow screw (9).

11. Prosthesis as claimed in claim 2, characterized in that the clamping sleeve (11) has on the outside and the tibial hollow screw (9) on the inside oblique cooperating slide-on faces (13).
